(19) United States
(12) Patent Application Publication
(10) Pub. No.: US 2025/0166849 A1
(43) Pub. Date: May 22, 2025

(54) INFECTION OUTBREAK ANALYSIS USING LONG-READ SEQUENCING DATA

(71) Applicant: Agency for Science, Technology and Research, Singapore (SG)

(72) Inventors: Niranjan Nagarajan, Singapore (SG); Chayaporn Suphavilai, Singapore (SG); Kwan Ki Ko, Singapore (SG); Kern Rei Chng, Singapore (SG); Kar Mun Lim, Singapore (SG)

(21) Appl. No.: 18/849,424

(22) PCT Filed: Mar. 9, 2023
(86) PCT No.: PCT/SG2023/050149

§ 371 Date: Sep. 20, 2024

(30) Foreign Application Priority Data
Mar. 23, 2022 (SG) 10202202958R

(51) Int. Cl. G16H 50/80; G16B 20/40; G16B 30/00; G16B 40/30

(52) U.S. Cl. CPC G16H 50/80; G16B 20/40; G16B 30/00; G16B 40/30

(57) ABSTRACT

Systems and methods for outbreak analysis by performing taxonomic classification of the first LRS data to assign one or more taxonomic identifiers to each record in the first LRS data; identifying a plurality of species-specific candidate genomes from a reference genome database based on the assigned taxonomic identifiers; aligning the first LRS data with each of the plurality of species-specific candidate genomes to determine a first species identity of a microorganism species present in the sample; identifying a first nearest strain of the identified microorganism species present in the first sample by aligning the first LRS data with a plurality of strain-specific candidate genome dataset.

### TECHNICAL FIELD

This disclosure generally relates to methods and systems for analysis of infection outbreaks using long-read sequencing data.

### BACKGROUND

This background description is provided to generally present the context of the disclosure. Contents of this background section are neither expressly nor impliedly admitted as prior art against the present disclosure.

Infections, including infections in clinical settings may have various origins. In addition, microbes causing the infections may have their respective evolution trajectories as they are spread. Infections may include healthcare-associated infections (HAI) that comprise infections that were not present or incubating in patients at the time of admission but were picked up by the patient during the process of care in a hospital or other healthcare facility. HAIs are associated with significant morbidity and excess mortality, prolonged hospital stay, long-term disability, increased antimicrobial resistance, excess deaths, and high healthcare costs. Given the transmissible nature and association with antimicrobial resistance, HAIs are of major public health concern. Effective HAI detection and mitigation can protect patients from harm associated with HAIs and allow scarce healthcare resources to be appropriated to other areas of need.

Conventional laboratory-based methods to delineate relatedness among bacterial isolates include pulse field gel electrophoresis (PFGE). Recently, molecular-based methods, such as next-generation sequencing (NGS) have been adopted for outbreak investigations. NGS data enables multilocus sequence typing (MLST) and genome assembly, a method to obtain ultra-long DNA sequences (supercontigs or chromosomes) for pathogen DNA/RNA comparison. However, conventional methods demand specialized equipment and expertise. Furthermore, the conventional methods are technically cumbersome, needing different restriction enzymes (PFGE), different sequencing primers for different species involved (MLST), or requiring high computational resources (genome assembly).

Although outbreaks are generally considered to be caused by the spread of bacteria strain(s), the rapid spread of AMR strains may be caused by horizontal gene transfer. No efficient method exists for the rapid analysis of possible plasmid outbreaks where a clinically important AMR gene spreads across species via horizontal gene transfer. With rising AMR globally, rapid analysis of both chromosomal and plasmid-borne AMR genes is critical to optimize antimicrobial use. When NGS is used for outbreak investigations, human bioinformatics expertise is required for sequence analysis. Due to these constraints, suspected outbreak samples are often submitted to reference laboratories for specialized analysis, resulting in delays in mitigation measures.

It is desired to address or ameliorate one or more disadvantages or limitations associated with the prior art, or to at least provide a useful alternative.

### SUMMARY

Some embodiments relate to a system for outbreak analysis, the system comprising one or more processing units to:
receive long-read nucleic acid sequence data (first LRS data) from a first sample;
perform taxonomic classification of the first LRS data to assign one or more taxonomic identifiers to each record in the first LRS data;
identify a plurality of species-specific candidate genomes from a reference genome database based on the assigned taxonomic identifiers;
align the first LRS data with each of the plurality of species-specific candidate genomes to determine a first species identity of a microorganism species present in the sample;
identify a first nearest strain of the identified microorganism species present in the first sample by aligning the first LRS data with a plurality of strain-specific candidate genome dataset.

Identifying a first nearest strain may comprise: aligning subset of the first LRS data corresponding to the first species with a plurality of candidate strains genomes of the first species; for each of the plurality of candidate strains genomes, calculating genomic overlap in relation to the subset of the first LRS data; and
identifying a candidate strain as the first nearest strain in the first sample based on a highest genomic overlap value.

In some embodiments, the one or more processing units are further configured to: monitor abundance of taxonomic identifiers assigned to the first LRS data; select a subset of species-specific candidate genomes from a reference genome database in response to the abundance of a subset of the taxonomic identifiers being stable.

In some embodiments, the one or more processing units are further configured to: receive long-read nucleic acid fragment sequence data (second LRS data) from a second sample; perform taxonomic classification of the second LRS data to assign one or more taxonomic identifiers to each record in the second LRS data; identify a subset of the second LRS data corresponding to the first species identity based on the assigned taxonomic classifiers; and align the second LRS data with the plurality of strain-specific candidate genome dataset to identify a second nearest strain in the second sample.

In some embodiments, the one or more processing units are further configured to determine a similarity or difference between the first and second nearest strains to identify an infection outbreak.

In some embodiments, the one or more processing units are further configured to populate a phylogenetic structure based on the determined similarity or difference between the first and second nearest strains.

In some embodiments, the one or more processing units are further configured to: align records of the first LRS data to determine a first contig, wherein the contig represents a chromosome or a plasmid of a microorganism in the first sample; align the first contig with a first draft genome by correcting alignment errors of records of the first LRS data to construct a first consensus genome; detect sequences of interest in the first consensus genome by comparing the first consensus genome with a database of known sequences of interest.

In some embodiments, the sequences of interest comprise Antimicrobial Resistance (AMR) genes or virulent factor sequences.

Comparing the consensus genome with the database of known sequences of interest may comprise determining an identity score, or a sequence coverage score of each record in the database of known sequences of interest with the consensus genome.

In some embodiments, the one or more processing units are further configured to: receive long-read nucleic acid fragment sequence data (second LRS data) from a second sample; align records of the second LRS data to determine a second contig, wherein the contig represents a chromosome or a plasmid of a microorganism in the second sample; align the second contig with a second draft genome by correcting alignment errors of records of the second LRS data to construct a second consensus genome; detect sequences of interest in the second consensus genome by comparing the second consensus genome with a database of known sequences of interest.

In some embodiments, the one or more processing units are further configured to compare the sequences of interest detected in the first consensus genome with the sequences of interest detected in the second consensus genome to detect a horizontal transfer of any one of the sequences of interest between microorganisms in the first and the second sample.

Some embodiments relate to a method for infection outbreak analysis, the method comprising: determining long-read nucleic acid sequence data (first LRS data) from a first sample; performing taxonomic classification of the first LRS data to assign one or more taxonomic identifiers to each record in the first LRS data; identifying a plurality of species-specific candidate genomes from a reference genome database based on the assigned taxonomic identifiers; aligning the first LRS data with each of the plurality of species-specific candidate genomes to determine a first species identity of a microorganism species present in the sample; identifying a first nearest strain of the identified microorganism species present in the first sample by aligning the first LRS data with a plurality of strain-specific candidate genome dataset.

The method of some embodiments further comprises subjecting the first sample to selective culture growth conditions to select specific groups of microorganisms for infection outbreak analysis.

The method of some embodiments further comprising: monitoring abundance of taxonomic identifiers assigned to the first LRS data; selecting a subset of species-specific candidate genomes in response to the abundance of a subset of the taxonomic identifiers being stable.

The method of some embodiments further comprises: determining long-read nucleic acid fragment sequence data (second LRS data) from a second sample; performing taxonomic classification of the second LRS data to assign one or more taxonomic identifiers to each record in the second LRS data; identifying a subset of the second LRS data corresponding to the first species identity based on the assigned taxonomic classifiers; and aligning the second LRS data with the plurality of strain-specific candidate genome dataset to identify a second nearest strain in the second sample.

The method of some embodiments further comprises determining a similarity or difference between the first and second nearest strains to identify an infection outbreak.

The method of some embodiments further comprises populating a phylogenetic structure based on the determined similarity or difference between the first and second nearest strains.

The method of some embodiments further comprises: aligning records of the first LRS data to determine a first contig, wherein the contig represents a chromosome or a plasmid of a microorganism in the first sample; aligning the first contig with a first draft genome by correcting alignment errors of records of the first LRS data to construct a first consensus genome; comparing the first consensus genome with a database of known sequences of interest to detect sequences of interest in the first consensus genome.

The sequences of interest may comprise Antimicrobial Resistance (AMR) genes or a virulent factor sequence.

Comparing the consensus genome with the database of known sequences of interest may comprise determining an identity score or a sequence coverage score of each record in the database of known sequences of interest with the consensus genome.

The method of some embodiments further comprises: determining long-read nucleic acid fragment sequence data (second LRS data) from a second sample; aligning records of the second LRS data to determine a second contig, wherein the second contig represents a chromosome or a plasmid of a microorganism in the second sample; aligning the second contig with a second draft genome by correcting alignment errors of records of the first LRS data to construct a second consensus genome; comparing the second consensus genome with a database of known sequences of interest to detect sequences of interest in the second consensus genome.

The method of some embodiments further comprises comparing the sequences of interest detected in the first consensus genome with the sequences of interest detected in the second consensus genome to detect a horizontal transfer of any one of the sequences of interest between microorganisms in the first and the second sample.

### BRIEF DESCRIPTION OF THE DRAWINGS

Exemplary embodiments of the present invention are illustrated by way of example in the accompanying drawings in which like reference numbers indicate the same or similar elements and in which:

FIG. 1 is a schematic diagram illustrating a part of a method according to the disclosure;

FIG. 2 is another schematic illustrating a part of a method according to the disclosure;

FIG. 3 is a schematic diagram illustrating a comparison between the methods according to the disclosure and baseline methods for outbreak tracing;

FIG. 4 illustrates a cluster map illustrating part of an outbreak tracing result obtained by an embodiment according to the disclosure; and

FIG. 5 illustrates a block diagram of a system for outbreak analysis.

### DETAILED DESCRIPTION

The embodiments provide systems and methods for rapid detection or tracking of potential outbreaks using sequence-based technology that can determine chromosomal or plasmid genetic relatedness. Some embodiments also identify chromosome or plasmid-borne AMR genes and other potentially pathogenic genes in samples. The embodiments include computer systems or platforms for providing analysis services for log-read sequencing data from the perspective of outbreak analysis. The systems or platforms may provide output in the form of an analytics report comprising: phylogenetic trees, strain/plasmid similarity maps, strain-specific cluster reporting to support the analysis of outbreaks in a facility such as a healthcare facility.

Rapid detection and mitigation of infections including HAIs requires both epidemiological data and laboratory data. When an outbreak is suspected, the laboratory performs cultures (from relevant clinical samples and environmental samples) and obtains bacterial isolates for downstream analysis to determine relatedness. Evaluation of the relationship between outbreak isolates is valuable for the understanding of the outbreak. The relationship between the outbreak isolates may allow the identification of previously unknown channels of transmission of infections in a healthcare setting. With the knowledge of the channels of transmission, the embodiments generate indications for taking preventative measures to control the spread of infection.

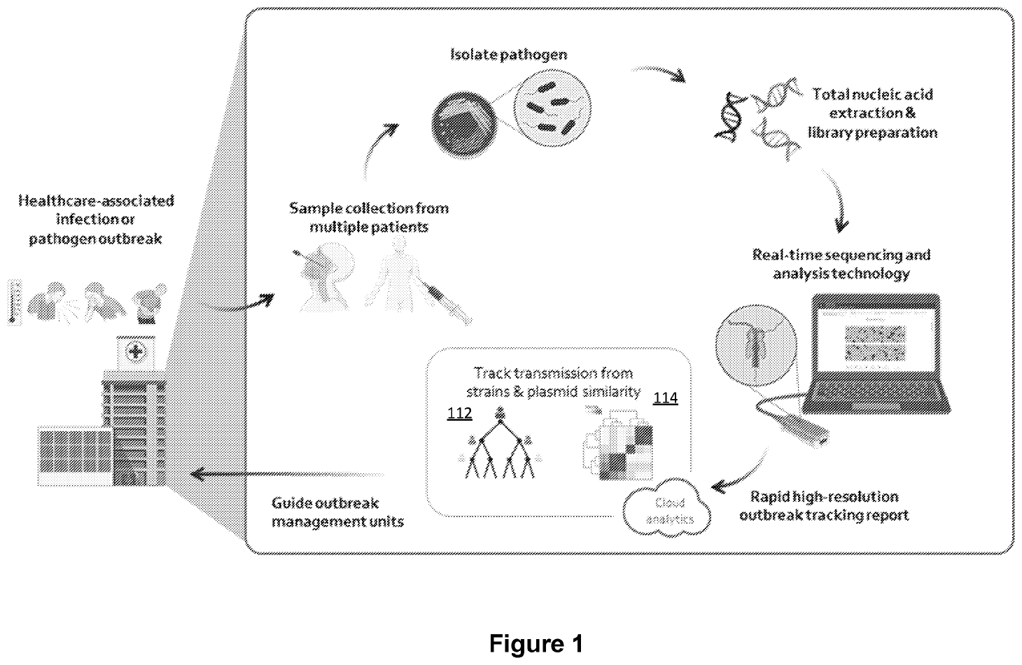

The embodiments enable outbreak analysis and/or tracking by providing computational models and methods that process long-read sequencing information (LRS data) obtained from the nanopore DNA sequencing platforms or other similar platforms that provide long-read nucleic acid data (LRS data). The algorithms of the embodiments may differentiate strains with 0.01% or lower average nucleotide difference, facilitating identification and/or differentiation of outbreak clusters. Some embodiments also identify Antimicrobial Resistance (AMR) genes on bacterial plasmids, gaining insight into the horizontal transfer of antimicrobial resistance properties across bacterial isolates. The embodiments provide DNA sequencing protocols and computational models for processing long-read data to provide insightful information for driving analysis of an outbreak in a clinical setting such as a hospital as illustrated in FIG. 1.

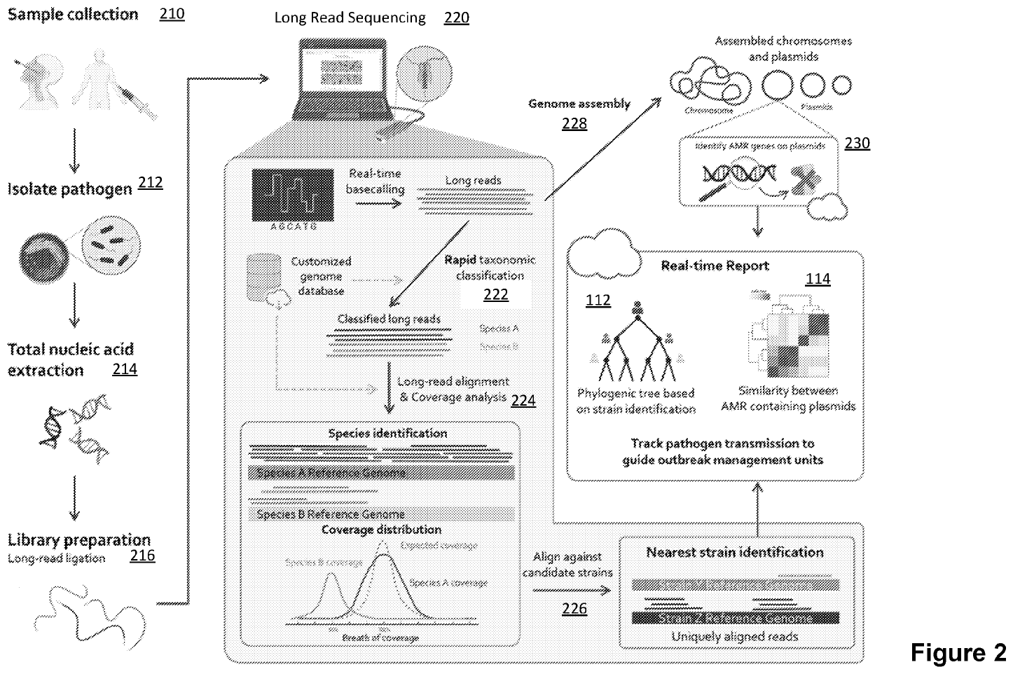

The embodiments may be integrated with laboratory or clinical protocols and the algorithms of the embodiments utilize long-read sequencing data to provide insightful information for outbreak analysis as illustrated in FIG. 2. Some embodiments have been described with reference with to a single sample or two samples. The methods and computational steps of the embodiments can be extended to process a plurality of samples obtained from a plurality of locations in a clinical setting. The outbreak analysis results, such as strain identification, strain specific clustering, comparison between two strains, analysis of strain/plasmid similarity, generation of similarity maps/phylogenetic trees, can be extended to a plurality of samples. The reference to a single sample, or a first/second sample is merely for convenience and is not intended to limit the scope of the claimed embodiments.

Obtaining Pathogen Isolates from Clinical Samples

Routine clinical samples, such as bronchoalveolar lavage (BAL), screening swabs and blood culture samples, etc. are collected from patients as per routine clinical practice (step 210 of FIG. 2). The samples are collected aseptically in sterile containers and transported to the onsite hospital diagnostic laboratory for processing. In some instances, the clinical samples may be processed within 1 hour of collection. The pathogens are isolated from the samples through a series of selective culture growth conditions to select specific groups of bacteria.

### DNA Extraction and Nanopore Library Preparation

The disclosed embodiments are applicable for any DNA sequencing platform that can generate sequencing data containing “reads” that are longer than 1,000 bases in length. Some embodiments use nanopore sequencing technology for generating sequencing data (first/second LRS data). Total nucleic acid extraction (step 214 of FIG. 2) and library preparation (step 216 of FIG. 2) may be performed as per nanopore long-read sequencing protocols (e.g. SQK-LSK109/LSK110). When MinION flow cells are used, a maximum of 24 or 96 samples (depending on the choice of barcoding kits) can be sequenced in a single run to generate the LRS data.

### Computational Modules for Pathogen Outbreak Tracking

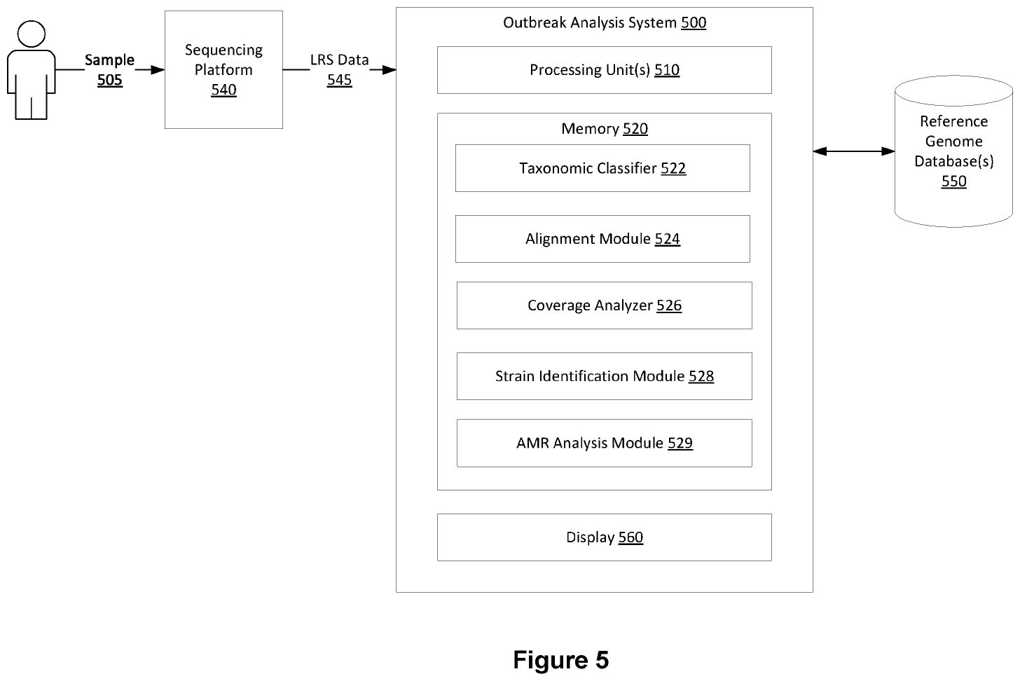

FIG. 5 illustrates an outbreak analysis system 500 and its associated components. Once the sequencing of the sample 505 begins (step 220 of FIG. 2), raw sequencing signals are locally converted into DNA sequences (reads) in real-time. The demultiplexed reads (LRS data 545) are streamed to the outbreak analysis system 500. This disclosure contemplates any suitable number of systems 500. This disclosure contemplates computer system 500 taking any suitable physical form. As example and not by way of limitation, computer system 500 may be an embedded computer system, a system-on-chip (SOC), a single-board computer system (SBC) (such as, for example, a computer-on-module (COM) or system-on-module (SOM)), a desktop computer system, a laptop or notebook computer system, a mainframe, a mesh of computer systems, a mobile telephone, a server, a tablet computer system, or a combination of two or more of these. Where appropriate, computer system 500 may include one or more computer systems 500; be unitary or distributed; span multiple locations; span multiple machines; span multiple data centers; or reside in a cloud, which may include one or more cloud components in one or more networks. Where appropriate, one or more computer systems 500 may perform without substantial spatial or temporal limitation one or more steps of one or more methods described or illustrated herein. One or more computer systems 500 may perform at different times or at different locations one or more steps of one or more methods described or illustrated herein, where appropriate. The system 500 allows automatic and scalable analysis of various numbers of samples at different time points. Two analysis modules of the embodiments are described below.

### Rapid Nearest Strain Identification Module

Memory 520 of the system 500 comprises program code to process the LRS data 545 generated by the sequencing platform 540. The streaming-in reads (raw sequencing signals, LRS data 545) are classified to a specific species using the taxonomic classifier 522 (at step 222 of FIG. 2) based on a curated pangenome database (reference genome database 550). Depending on the sequencing throughput, once the abundance of a species in the raw sequencing signals from a sample reaches a certain threshold (for example, the total number of nucleotides identified in the sample is equal to or greater than the genome size of a given species), the embodiment may retrieve or query a species-specific records from the reference genome database for subsequent analysis. The records of the LRS data are classified based on the pangenome and/or species-specific databases or both databases simultaneously. The taxonomic classifier monitors species specific abundance. When the abundance is stable 10-20 candidate genomes are retrieved from the reference genome database for long-read alignment. Abundance monitoring is performed by the system 500 based on the output of the taxonomic classifier as the LRS data is received by the system 500. Abundance monitoring over a prolonged analysis period helps to avoiding of the possible abundance biases at the beginning of the sequencing process and results in more accurate strain identification outcomes.

The processing of LRS data may be performed over several intervals. For each interval, the embodiments obtain LRS data that may be stored in a FASTQ format file, which contains multiple “reads”, i.e., DNA fragments with different lengths (1000-100,000 nucleotides). K-mer profile is extracted for each record in the LRS data. K-mer refers to all subsequences of a read with length K, where K ranges from 3 to 31 nucleotides.

The taxonomic classifier 522 assigns one or more taxonomic identifiers to each read based on K-mer profile and based on a comparison with records in the reference genome database 550 accessible to the taxonomic classifier 522. A taxonomic identifier represents an operational taxonomic unit (OTU), where the unit might refer to domain, kingdom, phylum, class, order, family, genus, species, strain, or individual genome. The reference genome database comprises DNA sequences of microbial genomes of interest for outbreak tracking. The taxonomic classifier incrementally updates the count for each OTU (species-level) as the LRS data is received by the system 500. The taxonomic classifier monitors the OTU count and triggers coverage analysis by the coverage analyzer 526 for each species when the species count passes a threshold. The species with a count higher than the threshold in an iteration may be referred to as candidate species. The threshold may be defined based on the total number of sequenced nucleotides and the genome size of each species.

The alignment module 524 performs alignment of the LRS data associated with the candidate species with the associated species-specific reference genomes. Based on the long-read alignment results, coverage analysis is performed by the coverage analyzer 526 to confirm the presence of one or more species in the sample 505.

The system 500 selects candidate reference genomes for each species based on the OTU count at strain or individual genome level. The alignment module align the classified LRS data to the candidate reference genomes and records genomic regions that are covered by the reads. A read may be said to align with a genome if an identity score (total number of matched nucleotides/alignment length×100) is at least 80% or 85% or 90% and/or a read-coverage score (alignment length/read length×100) is at least 80% or 85% or 90%. Given the alignment records, the coverage analyzer calculates the percentage of the breadth of coverage for each of the candidate species. The coverage analyzer may confirm the presence of the species when the coverage percentage is at least 40-90% of the expected coverage of each species. The expected coverage percentage may follow a Poisson distribution, which takes into account the total number of sequenced nucleotides and the genome size of each species. In alternative embodiments, other suitable distributions may be used by the coverage analyzer, including a negative binomial distribution.

### Strain Level Taxonomic Classification

The long-read alignment also enables the identification of unique regions in the LRS data that provide the basis for determining the nearest strain (step 226 of FIG. 2) to the microorganism identified in the sample. As the LRS data is received by the system 500, the system 500 updates the species and/or strain identification results in real-time or near-real time. The generated results may be presented on a display 560.

For each detected species, the embodiments retrieve reference genomes and calculate genome similarity based on whole-genome information. The reference genomes are then grouped based on similarity values. The similarity values in some embodiments may be: 99%, 99.5%, 99.9%, 99.95%, or 99.99%. A representative genome from the reference genomes is selected for each cluster/group defined based on the similarity values. A species-specific dataset is defined based on the selected representative genomes for taxonomic classification. Taxonomic classification in relation to the species-specific dataset is performed as explained above, however, the OTU used for taxonomic classification are strain-level OTUs or associated with a particular known strain of a microorganism. The number of candidate genomes in the strain-specific dataset may be less than or equal to 10, 15 or 20. The reduction in the search space of the candidate genomes in the strain-specific dataset based on the earlier coverage analysis using the pan-genome records in the reference genome database advantageously reduces the computational complexity of the strain identification operations. Based on the alignment results from the coverage analysis operations, the embodiments identify reads that are uniquely aligned to single candidate reference genomes in the strain-specific dataset.

For each candidate representative genome in the strain-specific dataset, the embodiment determines a percentage of the genomic region that is covered by the uniquely aligned reads (also referred to as genomic overlap). The system 500 then compares the genomic overlap values from all candidate representative genomes and identifies the nearest strain, i.e., the candidate representative genome with the highest percentage of overlap. The nearest strain is said to be significantly matched if the highest percentage is significantly higher than the average overlap percentage across all candidate representative genomes.

### Detecting and Locating AMR Genes

The system 500 may also comprise an AMR gene analysis module 529. The AMR gene analysis module performs genome assembly (step 228 of FIG. 2) and polishing to obtain high-quality draft genomes that consist of full-length (or nearly full-length) chromosome and plasmid sequences. The assembly process may be started when all samples reach a certain sequencing depth (>50-100×) or after the sequencing is completed. All assembled sequences are then aligned using a pairwise genome alignment method. The reads are aligned to draft genomes and/or an AMR gene databases part of the reference genome database 550. The AMR analysis module then locates AMR genes in the LRS data and reports a chromosome or a plasmid that harbours the AMR genes as illustrated in the example of Table 1.

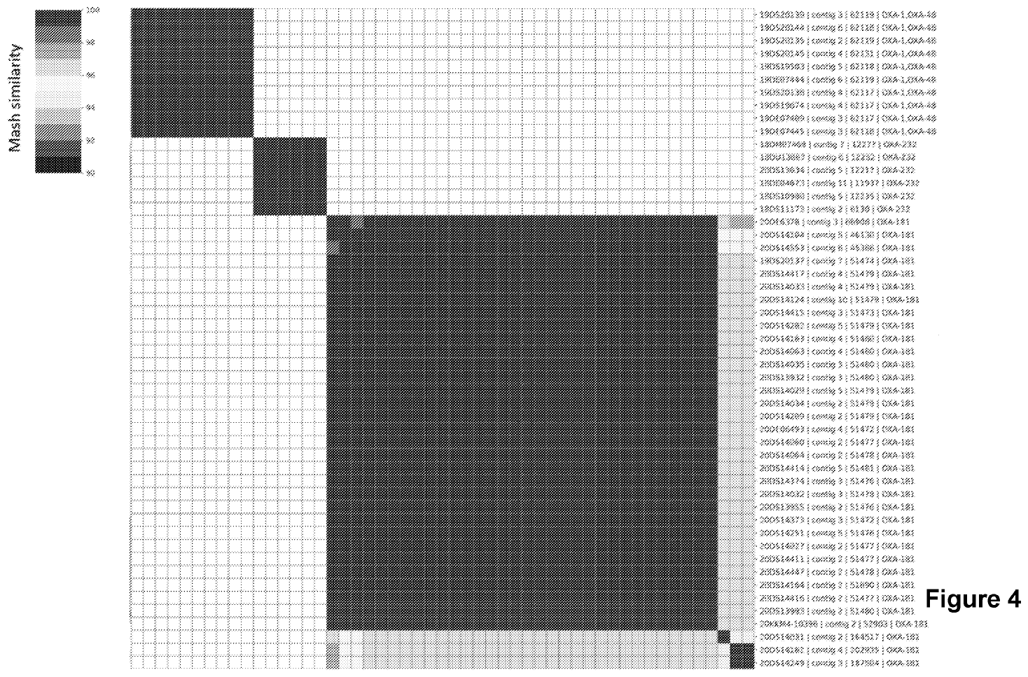

By integrating pairwise alignment of plasmids and AMR gene alignment results, the embodiments allow pinpointing a plasmid containing the AMR gene of interest (for example an input by a user of the system 500; such as OXA family). An AMR containing plasmid similarity dendrogram is generated by the system 500, providing insight into horizontal plasmid transfer within an outbreak as illustrated in FIG. 4.

Long reads obtained from samples (received in a fastq format, for example) are aligned with each other and the concatenation (i.e. a contig) of all reads is reported or identified by the system 500. Each contig may represent a chromosome or plasmid. There could be more than one contig associated with a sample when there are plasmids in the isolate, or when the genome assembly process might fail to align all chromosomal reads into a single contig. A single contig or a set of contigs from each isolate sample as are referred to as a draft genome.

The embodiment aligns a subset or all long reads obtained from a sample to the draft genome and constructs a consensus genome. Any errors in the LRS data are corrected in the assembly step. A polishing step (alignment of all long reads) can be done more than once to reduce errors. The embodiment determines a consensus genome, generated. Contig polishing comprises resolving putative assembly errors in the contigs by mapping the LRS data originating from a sample to the contigs and building a consensus of this read mapping.

The system 500 identifies whether each contig is from chromosome or plasmid by inspecting mobile/plasmid elements within the contig. Based on chromosomal contigs from multiple isolates/samples, whole chromosome comparison or phylogenetic analysis may be performed by the system 500. Based on contigs originating from plasmids from multiple isolates/samples, whole plasmid comparison or phylogenetic analysis may also be performed. Phylogenetic analysis comprises generation of a phylogenetic structure/tree (112 in FIGS. 1, 2, FIG. 3) illustrating evolutionary relationships between a plurality of strains identified from several samples. Phylogenetic analysis may also comprise analysis of the similarity between AMR containing plasmids sequenced and identified from a plurality of samples. The AMR similarity analysis may be represented in the form of a similarity heatmap (114 in FIGS. 1, 2).

The embodiments detect AMR genes and virulent factors (i.e., sequences of interest) in each polished contig by aligning AMR and virulent factor sequences onto the contig. Open reading frames can also be identified as part of the step of detection of a sequence of interest. The sequence of interest may be considered to align with an AMR gene if an identity score is at least 90%, 95% or 99% and/or a sequence-coverage score (alignment length/sequence length×100) is at least 80%, 85% or 90%, for example.

Based on AMR genes detected in plasmids, horizontal gene transfer can be detected in the event that the same AMR gene is detected in different isolates. Long reads might also be directly aligned to the sequences of interest by the system 500. A read is said to align with an AMR gene if an identity score is at least 80%, 85% or 90% and a sequence-coverage score (alignment length/gene length×100) is at least 80%, 85% or 90%. The embodiment may reports a list of AMR genes, virulent factors, horizontal gene transfer events detected within the input isolate. Horizontal gene transfer (HGT) comprises is the non-sexual movement of genetic information between genomes of microorganisms in two samples. HGT includes the spread of antibiotic resistance genes among bacteria and detection of HGT across samples provides an insight into the outbreak on an infection in a facility. Detection of HGT allows the evaluation of effectiveness of modes of treatment and implementation of a change in modes of treatment or infecting control mechanisms if necessary.

Comparison with Different Technologies for Outbreak Tracking

The results obtained using the embodiments were compared against existing outbreak analysis techniques, using 52 Klebsiella pneumoniae isolates obtained between 2018-2020 from both clinical and environmental samples. FIG. 2 illustrates a schematic diagram of a flowchart of nearest strain identification and outbreak tracking according to an embodiment. Existing outbreak tracking techniques including Pulsed-Field Gel Electrophoresis (PFGE) and Next Generation Sequencing (NGS) based on the Illumina platform were used as a baseline for comparison. NGS data were analyzed using two baseline methods:

NGS-MLST: Multi-Locus Sequence Typing (MLST) compares the Illumina short reads to the standard typing scheme provided by PubMLST (Jolley, Bray & Maiden, 2018) and then identifies Sequence Typing (ST) using SRST2 (Inouye et al., 2014).

NGS-ASM: Draft genomes are assembled using the Illumina short reads (GIS's GERMS pipeline). A phylogenetic tree is created based on the distance between the core genome of multiple isolates (Treangen, Ondov, Koren & Phillippy, 2014).

All baseline methods suggest distinct outbreak clusters across different years. NGS-MLST and NGS-ASM provide similar outbreak cluster information, while NGS-MLST provided failed to specifically cluster several samples. The nearest strains identified based on the disclosed embodiments were consistent with PFGE based results.

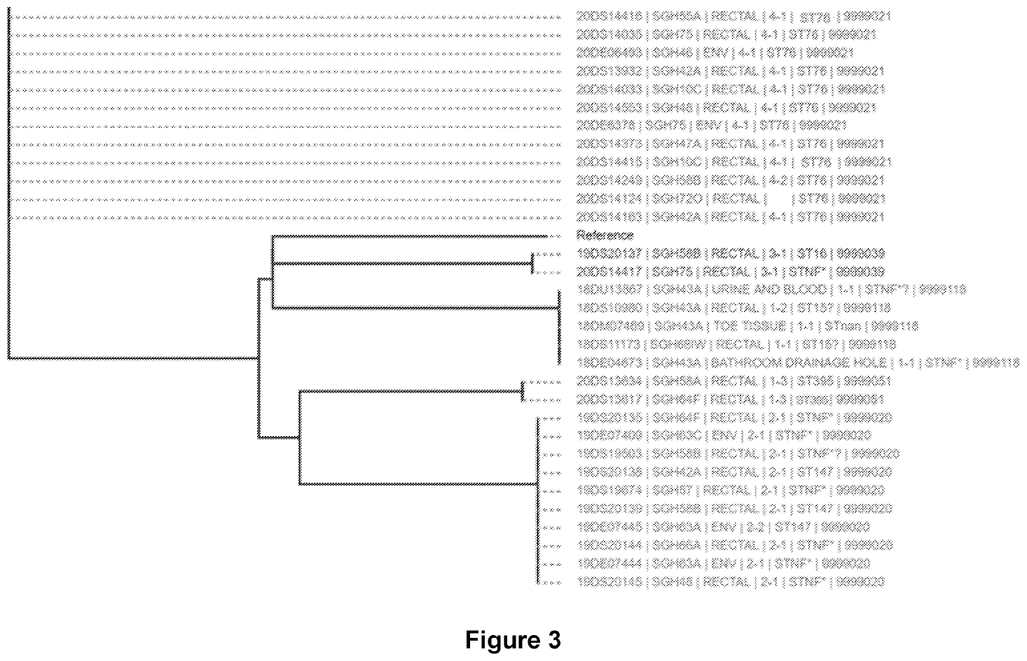

FIG. 3 illustrates a comparison between different outbreak tracking techniques. The phylogenetic tree of FIG. 3 was constructed based on NGS-ASM. Each node represents a Klebsiella pneumoniae isolate, where the label describes a year of collection, location, sample site, PFGE type, MLST (NF=Not Found; *=mismatches; ?=uncertainty; nan=no core gene alignment), and the nearest strain ID identified by the disclosed embodiment (9999XXX). As illustrated in FIG. 3, the strain classification generated by the embodiments (9999XXX associated with each isolate) is in line with the phylogenetic tree generated based on NGS-ASM.

In addition to the sequencing analyses based on core-genome/chromosomal information, advantage of some embodiments is to provide the ability to assess whether AMR genes are spreading within an outbreak. It was observed in experiments that the majority of the AMR gene families were on plasmids and could be transferable to other bacteria. While PFGE and NGS-based methods do not allow detection of the transferable plasmids, the AMR analysis module of system 500 could identify and locate AMR genes in samples. The similarity between plasmids across different bacteria isolates was calculated, providing unique high-resolution information for outbreak tracking as exemplified from the results illustrated in FIG. 4. An experiment detected and located OXA-48 like genes on a plasmid in 51 isolates (out of 52 isolates where OXA-48 like genes were detected by qPCR). As expected, the majority of the cases in the experiment detected unique plasmid types in different years.

FIG. 4 illustrates a cluster map that presents similarities between plasmids containing AMR gene(s) across 51 Klebsiella pneumoniae isolates. Each row label contains sample ID, contig ID, contig/plasmid size, and the detected OXA genes. The shade represents plasmid similarity. The system 500 may generate such similarity heat maps and present the generated heat map on display 560.

Integration into Routine Outbreak Tracking in a Hospital

An integrated result according to some embodiments consists of outbreak insights based on real-time analysis for nanopore long-read sequencing data in Module 1 and Module 2 as illustrated in Table 1 below highlights different outbreak perspectives provided by the results of the embodiments.

TABLE 1
An integrated result

| | | Contig | AMR | Plasmids | Tax ID |
|---|---|---|---|---|---|
| Sample ID | Contig ID | size | genes | (PlasmidFinder 2.0) | (Rapid) |
| 19DS20139 | contig_3 | 62119 | OXA-1, | IncM1_1 (99.46%) | 9999020 |
| | | | OXA-48 | | |
| 19DS20144 | contig_6 | 62118 | OXA-1, | IncM1_1 (99.46%) | 9999020 |
| | | | OXA-48 | | |
| 19DS20135 | contig_2 | 62119 | OXA-1, | IncM1_1 (99.46%) | 9999020 |
| | | | OXA-48 | | |
| 19DS20145 | contig_4 | 62131 | OXA-1, | IncL_1 (100.00%), | 9999020 |
| | | | OXA-48 | IncM1_1 (100.00%), | |
| | | | | IncM2_1 (98.90%) | |
| 19DS19503 | contig_5 | 62118 | OXA-1, | IncM1_1 (99.46%) | 9999020 |
| | | | OXA-48 | | |
| 19DE07444 | contig_6 | 62119 | OXA-1, | IncM1_1 (99.46%) | 9999020 |
| | | | OXA-48 | | |
| 19DS20138 | contig_4 | 62117 | OXA-1, | IncM1_1 (99.46%) | 9999020 |
| | | | OXA-48 | | |
| 19DS19674 | contig_4 | 62117 | OXA-1, | IncM1_1 (99.46%) | 9999020 |
| | | | OXA-48 | | |
| 19DE07409 | contig_3 | 62117 | OXA-1, | IncM1_1 (99.46%) | 9999020 |
| | | | OXA-48 | | |
| 19DE07445 | contig_3 | 62118 | OXA-1, | IncM1_1 (99.32%) | 9999020 |
| | | | OXA-48 | | |
| 18DM07469 | contig_7 | 12277 | OXA-232 | ColKP3_1 (100.00%) | 9999118 |
| 18DU13867 | contig_6 | 12252 | OXA-232 | ColKP3_1 (100.00%) | 9999118 |
| 20DS13634 | contig_5 | 12217 | OXA-232 | ColKP3_1 (100.00%) | 9999051 |
| 18DE04673 | contig_11 | 11937 | OXA-232 | ColKP3_1 (100.00%) | 9999118 |
| 18DS10980 | contig_5 | 12235 | OXA-232 | ColKP3_1 (100.00%) | 9999118 |
| 18DS11173 | contig_2 | 6130 | OXA-232 | ColKP3_1 (99.64%) | 9999118 |
| 20DE6378 | contig_3 | 66908 | OXA-181 | ColKP3_1 (100.00%), | 9999021 |
| | | | | IncX3_1 (100.00%) | |
| 20DS14104 | contig_5 | 46130 | OXA-181 | ColKP3_1 (100.00%), | 9999021 |
| | | | | IncX3_1 (100.00%) | |
| 20DS14553 | contig_6 | 45386 | OXA-181 | ColKP3_1 (100.00%) | 9999021 |
| 19DS20137 | contig_7 | 51474 | OXA-181 | ColKP3_1 (100.00%), | 9999039 |
| | | | | IncX3_1 (100.00%) | |
| 20DS14417 | contig_4 | 51479 | OXA-181 | ColKP3_1 (100.00%), | 9999039 |
| | | | | IncX3_1 (100.00%) | |
| 20DS14033 | contig_4 | 51479 | OXA-181 | ColKP3_1 (100.00%), | 9999021 |
| | | | | IncX3_1 (100.00%) | |
| 20DS14124 | contig_10 | 51479 | OXA-181 | ColKP3_1 (100.00%), | 9999021 |
| | | | | IncX3_1 (100.00%) | |
| 20DS14415 | contig_3 | 51473 | OXA-181 | ColKP3_1 (100.00%), | 9999021 |
| | | | | IncX3_1 (100.00%) | |
| 20DS14282 | contig_5 | 51479 | OXA-181 | ColKP3_1 (100.00%), | 9999021 |
| | | | | IncX3_1 (100.00%) | |
| 20DS14163 | contig_4 | 51480 | OXA-181 | ColKP3_1 (100.00%), | 9999021 |
| | | | | IncX3_1 (100.00%) | |
| 20DS14063 | contig_4 | 51480 | OXA-181 | ColKP3_1 (100.00%), | 9999021 |
| | | | | IncX3 1 (100.00%) | |
| 20DS14035 | contig_3 | 51480 | OXA-181 | ColKP3_1 (100.00%), | 9999021 |
| | | | | IncX3_1 (100.00%) | |
| 20DS13932 | contig_3 | 51480 | OXA-181 | ColKP3_1 (100.00%), | 9999021 |
| | | | | IncX3_1 (100.00%) | |
| 20DS14029 | contig_5 | 51479 | OXA-181 | ColKP3_1 (100.00%), | 9999021 |
| | | | | IncX3_1 (100.00%) | |
| 20DS14034 | contig_2 | 51479 | OXA-181 | ColKP3_1 (100.00%), | 9999021 |
| | | | | IncX3_1 (100.00%) | |
| 20DS14209 | contig_2 | 51479 | OXA-181 | ColKP3_1 (100.00%), | 9999021 |
| | | | | IncX3_1 (100.00%) | |
| 20DE06493 | contig_4 | 51472 | OXA-181 | ColKP3_1 (100.00%), | 9999021 |
| | | | | IncX3_1 (100.00%) | |
| 20DS14060 | contig_2 | 51477 | OXA-181 | ColKP3_1 (100.00%), | 9999021 |
| | | | | IncX3_1 (100.00%) | |
| 20DS14064 | contig_2 | 51478 | OXA-181 | ColKP3_1 (100.00%), | 9999021 |
| | | | | IncX3_1 (100.00%) | |
| 20DS14414 | contig_5 | 51481 | OXA-181 | ColKP3_1 (100.00%), | 9999021 |
| | | | | IncX3_1 (100.00%) | |
| 20DS14374 | contig_3 | 51476 | OXA-181 | ColKP3_1 (100.00%), | 9999021 |
| | | | | IncX3_1 (100.00%) | |
| 20DS14032 | contig_3 | 51478 | OXA-181 | ColKP3_1 (100.00%), | 9999021 |
| | | | | IncX3_1 (100.00%) | |
| 20DS13955 | contig_2 | 51476 | OXA-181 | ColKP3_1 (100.00%), | 9999021 |
| | | | | IncX3_1 (100.00%) | |
| 20DS14373 | contig_3 | 51472 | OXA-181 | ColKP3_1 (100.00%), | 9999021 |
| | | | | IncX3_1 (100.00%) | |
| 20DS14251 | contig_5 | 51476 | OXA-181 | ColKP3_1 (100.00%), | 9999021 |
| | | | | IncX3_1 (100.00%) | |
| 20DS14027 | contig_2 | 51477 | OXA-181 | ColKP3_1 (100.00%), | 9999021 |
| | | | | IncX3_1 (100.00%) | |
| 20DS14411 | contig_2 | 51477 | OXA-181 | ColKP3_1 (100.00%), | 9999021 |
| | | | | IncX3_1 (100.00%) | |
| 20DS14447 | contig_2 | 51478 | OXA-181 | ColKP3_1 (100.00%), | 9999021 |
| | | | | IncX3_1 (100.00%) | |
| 20DS14164 | contig_2 | 51690 | OXA-181 | ColKP3_1 (100.00%), | 9999021 |
| | | | | IncX3_1 (100.00%) | |
| 20DS14416 | contig_2 | 51477 | OXA-181 | ColKP3_1 (100.00%), | 9999021 |
| | | | | IncX3_1 (100.00%) | |
| 20DS13993 | contig_2 | 51480 | OXA-181 | ColKP3_1 (100.00%), | 9999021 |
| | | | | IncX3_1 (100.00%) | |
| 20KKM4- | contig_2 | 52903 | OXA-181 | ColKP3_1 (100.00%), | 9999021 |
| 10396 | | | | IncX3_1 (100.00%) | |
| 20DS14031 | contig_2 | 164517 | OXA-181 | ColKP3_1 (100.00%), | 9999021 |
| | | | | IncFIB(pKPHS1)_1 | |
| | | | | (100.00%), | |
| | | | | IncX3_1 (100.00%) | |
| 20DS14162 | contig_4 | 202935 | OXA-181 | ColKP3 1 (100.00%), | 9999021 |
| | | | | IncFIB(K)_1 (98.93%), | |
| | | | | IncX3_1 (100.00%) | |
| 20DS14249 | contig_3 | 187504 | OXA-181 | ColKP3_1 (100.00%), | 9999021 |
| | | | | IncFIB(K)_1 (98.93%), | |
| | | | | IncX3_1 (100.00%) | |

Some embodiments rely on both a cloud computing/remote computing platform (for highly scalable, real-time whole-genome analysis) and a local computer in a laboratory for live base calling (converting raw sequencing signal to DNA sequences). In a setup according to some embodiments, where 24 isolates were sequenced on a MinION flow cell, an experiment could identify the nearest strain for each isolate (Rapid Tax ID column) within 1-2 hrs and characterize OXA-containing plasmids within a day. The exemplary hardware and software specifications, as well as reagents, are listed in Table 2.

TABLE 2
Hardware and software specifications

Wet-lab related consumables
Sample collection consumables (e.g. swabs and Amies transport medium)
Bacteria culture media (non-selective and selective)
Bacteria culture consumables e.g. culture tubes, petri dish
Extraction kit e.g. Qiagen Powersoil Pro kit
Beckman Coulter AMPure XP
Oxford Nanopore Technologies Minion (FLO-MIN106D) or Flongle (FLO-
FLG001) flowcells
Oxford Nanopore Technologies SQK-LSK109/LSK110 kit with NEBNext ®
Companion Module. If intend to multiplex, NBD104 and NDB114, or NDB196
Wet-lab related equipment
Incubator for culturing (able to set a range of temperatures and shaking speed)
Centrifuge with 50 ml tube holders and 2 mL tube holders
Vortex with 2 ml tube adapters
Table-top centrifuge
DynaMag magnetic tube rack (for magnetic bead cleanups)
Oxford Nanopore Technologies Minion Mk1b or Mk1c. If intend to use Flongles
flowcells, Flongle adapter
Equipment and software for required in a lab
A workstation computer or laptop
8-core CPU
16-32 GB RAM
1 TB SSD
Graphical Processing Unit (GPU) with at least 8 GB memory
Required softwares
MinKNOW for nanopore sequencing
Guppy for live basecalling
Cloud computing resources
AWS EC2 machine with 32 GB RAM and 16-core CPU.
Required softwares
Python 3.x for real-time analysis scripts
MinKNOW for nanopore sequencing
Guppy for live basecalling
Minimap2 for long-read alignment
Kraken2 for taxonomic classification
Flye
MUMMer4
Mash

It will be appreciated that many further modifications and permutations of various aspects of the described embodiments are possible. Accordingly, the described aspects are intended to embrace all such alterations, modifications, and variations that fall within the spirit and scope of the appended claims.

Long-read nucleic acid fragment sequence (LRS) data includes sequencing data of at least 1,000 base pairs or more of a DNA or an RNA molecule. The long-read nucleic acid fragment sequence data may be obtained using nanopore sequencing or PacBio sequencing or any other long-read sequencing technique.

Predefined abundance level comprises a level of abundance considered statistically significant from the perspective of identification of a microorganism in a sample. The predefined abundance level may include a level wherein the total number of bases sequenced in a sample is equal to or greater than the genome size of a given species.

Reference genomes include genomes corresponding to a variety of species that may be potentially present in a sample. Reference genomes may be stored in a genome database populated by routine clinical analysis of samples by total nucleic acid extraction and long-read ligation. Candidate genomes include a subset of the reference genomes that are selected for coverage analysis. The candidate genomes are selected based on the taxonomic identifiers assigned to LRS data obtained from a sample. The selection of candidate genomes advantageously avoids the need for alignment of a large volume of LRS data with a large number of reference genomes making the methods of the embodiments computationally expensive. The candidate genomes may also be referred to as representative genomes or candidate reference genomes.

Aligning the LRS data with the candidate genomes includes matching nucleotides of the LRS data with the genome. Alignment can be measured or quantified by an identity score that may be defined as

$$\frac{total number of matched nucleotides}{alignment length}$$

or a read-coverage score defined as

$$\frac{alignment length}{read length} .$$

Coverage analysis comprises the calculation of the percentage of the breadth of coverage for each candidate genome based on the alignment results.

Species identity comprises genomic data or part of genomic data sufficient to uniquiely identify a distinct species of a microorganism in a sample.

Some embodiments are directed to methods/systems for outbreak analysis by: determining long-read nucleic acid fragment sequence data (LRS data) from a plurality of samples; performing taxonomic classification of the LRS data and comparison with a plurality of candidate genomes to determine a plurality of species present in the plurality of samples; aligning the LRS data of the plurality of species with a candidate strain genome database to determine a nearest strain of each of the plurality of species; determining a similarity or difference between the determined nearest strains to identify an infection outbreak.

Some embodiments are directed to systems/methods for determining a consensus genome by: determining long-read nucleic acid fragment sequence data (LRS data) from a sample; aligning records of the LRS data to determine one or more than one contigs, wherein the contig represents a chromosome or a plasmid; aligning the contigs with a draft genome by correcting alignment errors of the records of the LRS data to construct a consensus genome of a microorganism of the sample.

Some embodiments relate to systems/methods for detecting sequences of interest in a microorganism, the method comprising: determining long-read nucleic acid fragment sequence data (first LRS data) from a first sample; aligning records of the first LRS data to determine a first at least one contig, wherein the contig represents a chromosome or a plasmid of a microorganism in the first sample; aligning the first at least one contig with a first draft genome by correcting alignment errors of records of the first LRS data to construct a first consensus genome; comparing the first consensus genome with a database of known sequences of interest to detect sequences of interest in the first consensus genome.

Some embodiments relate to methods for treating infection by one or more microorganisms in a subject, the method comprising: determining long-read nucleic acid fragment sequence data (LRS data) from a sample; performing taxonomic classification of the LRS data to assign one or more taxonomic identifiers to each record on the LRS data; identifying a plurality of candidate genomes based on the taxonomic identifiers assigned to each record on the LRS data; aligning the LRS data with each of the plurality of candidate genomes to determine a species identity of a microorganism present in the sample; identifying a nearest strain to the microorganism based on the aligned LRS data and a candidate strain genome database; administering a therapeutic agent to the subject based on the identified nearest strain.

Some embodiments relate to systems/methods for outbreak tracking by: determining long-read nucleic acid fragment sequence data (LRS data) from a plurality of samples; performing taxonomic classification of the LRS data and comparison with a plurality of candidate genomes to determine a plurality of species present in the plurality of samples; aligning the LRS data of the plurality of species with a candidate strain genome database to determine a nearest strain of each of the plurality of species; determining a similarity or difference between the determined nearest strains to identify an infection outbreak; aligning records of the first LRS data to determine at least one contig, wherein the contig represents a chromosome or a plasmid of a microorganism in one of the plurality of samples; aligning the at least one contig with a draft genome by correcting alignment errors of records of the LRS data to construct a consensus genome; comparing the consensus genome with a database of known sequences of interest to detect sequences of interest in the consensus genome.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A system for outbreak analysis, the system comprising one or more processing units, the one or more processing units configured to:
receive long-read nucleic acid sequence data (first LRS data) from a first sample;
perform taxonomic classification of the first LRS data to assign one or more taxonomic identifiers to each record in the first LRS data;
identify a plurality of species-specific candidate genomes from a reference genome database based on the assigned taxonomic identifiers;
align the first LRS data with each of the plurality of species-specific candidate genomes to determine a first species identity of a microorganism species present in the sample; and
identify a first nearest strain of the identified microorganism species present in the first sample by aligning the first LRS data with a plurality of strain-specific candidate genome dataset.

2-23. (canceled)

24. The system of claim 1, wherein identifying a first nearest strain comprises:
aligning subset of the first LRS data corresponding to the first species with a plurality of candidate strains genomes of the first species;
calculating, for each of the plurality of candidate strains genomes, a genomic overlap in relation to the subset of the first LRS data; and
identifying a candidate strain as the first nearest strain in the first sample based on a highest genomic overlap value.

25. The system of claim 1, wherein the one or more processing units are further configured to:
monitor an abundance of taxonomic identifiers assigned to the first LRS data; and
select a subset of species-specific candidate genomes from a reference genome database in response to the abundance of a subset of the taxonomic identifiers being stable.

26. The system of claim 1, wherein the one or more processing units are further configured to:
receive long-read nucleic acid fragment sequence data (second LRS data) from a second sample;
perform taxonomic classification of the second LRS data to assign one or more taxonomic identifiers to each record in the second LRS data;
identify a subset of the second LRS data corresponding to the first species identity based on the assigned taxonomic classifiers; and
align the second LRS data with the plurality of strain-specific candidate genome dataset to identify a second nearest strain in the second sample.

27. The system of claim 26, wherein the one or more processing units are further configured to determine a similarity or difference between the first and second nearest strains to identify an infection outbreak.

28. The system of claim 26, wherein the one or more processing units are further configured to populate a phylogenetic structure based on the determined similarity or difference between the first and second nearest strains.

29. The system of claim 1, wherein the one or more processing units are further configured to:
align records of the first LRS data to determine a first contig, wherein the first contig represents a chromosome or a plasmid of a microorganism in the first sample;
align the first contig with a first draft genome by correcting alignment errors of records of the first LRS data to construct a first consensus genome; and
detect sequences of interest in the first consensus genome by comparing the first consensus genome with a database of known sequences of interest.

30. The system of claim 29, wherein the sequences of interest comprise Antimicrobial Resistance (AMR) genes or virulent factor sequences.

31. The system of claim 29, wherein comparing the consensus genome with the database of known sequences of interest comprises determining an identity score, or a sequence coverage score, of each record in the database of known sequences of interest with the consensus genome.

32. The system of claim 29, wherein the one or more processing units are further configured to:
receive long-read nucleic acid fragment sequence data (second LRS data) from a second sample;
align records of the second LRS data to determine a second contig, wherein the contig represents a chromosome or a plasmid of a microorganism in the second sample;
align the second contig with a second draft genome by correcting alignment errors of records of the second LRS data to construct a second consensus genome;
detect sequences of interest in the second consensus genome by comparing the second consensus genome with the database of known sequences of interest.

33. The system of claim 32, wherein the one or more processing units are further configured to compare the sequences of interest detected in the first consensus genome with the sequences of interest detected in the second consensus genome to detect a horizontal transfer of any one of the sequences of interest between microorganisms in the first and the second sample.

34. A method for infection outbreak analysis, the method comprising:
determining long-read nucleic acid sequence data (first LRS data) from a first sample;
performing taxonomic classification of the first LRS data to assign one or more taxonomic identifiers to each record in the first LRS data;
identifying a plurality of species-specific candidate genomes from a reference genome database based on the assigned taxonomic identifiers;
aligning the first LRS data with each of the plurality of species-specific candidate genomes to determine a first species identity of a microorganism species present in the sample; and
identifying a first nearest strain of the identified microorganism species present in the first sample by aligning the first LRS data with a plurality of strain-specific candidate genome dataset.

35. The method of claim 34, wherein identifying the first nearest strain comprises:
aligning subset of the first LRS data corresponding to the first species with a plurality of candidate strains genomes of the first species;
calculating, for each of the plurality of candidate strains genomes, a genomic overlap in relation to the subset of the first LRS data; and
identifying a candidate strain as the first nearest strain in the first sample based on a highest genomic overlap value.

36. The method of claim 34, wherein the method further comprises subjecting the first sample to selective culture growth conditions to select specific groups of microorganisms.

37. The method of claim 34, further comprising:
monitoring an abundance of taxonomic identifiers assigned to the first LRS data; and
selecting a subset of species-specific candidate genomes in response to the abundance of a subset of the taxonomic identifiers being stable.

38. The method of claim 34, further comprising:
determining long-read nucleic acid fragment sequence data (second LRS data) from a second sample;
performing taxonomic classification of the second LRS data to assign one or more taxonomic identifiers to each record in the second LRS data;
identifying a subset of the second LRS data corresponding to the first species identity based on the assigned taxonomic classifiers; and
aligning the second LRS data with the plurality of strain-specific candidate genome dataset to identify a second nearest strain in the second sample.

39. The method of claim 38, further comprising determining a similarity or difference between the first and second nearest strains to identify an infection outbreak.

40. The method of claim 38, further comprising populating a phylogenetic structure based on the determined similarity or difference between the first and second nearest strains.

41. The method of claim 34, further comprising:
aligning records of the first LRS data to determine a first contig, wherein the contig represents a chromosome or a plasmid of a microorganism in the first sample;
aligning the first contig with a first draft genome by correcting alignment errors of records of the first LRS data to construct a first consensus genome; and
comparing the first consensus genome with a database of known sequences of interest to detect sequences of interest in the first consensus genome.

42. The method of claim 41, wherein the sequences of interest comprise Antimicrobial Resistance (AMR) genes or a virulent factor sequence.

43. The method of claim 41, wherein comparing the consensus genome with the database of known sequences of interest comprises determining an identity score, or a sequence coverage score, of each record in the database of known sequences of interest with the consensus genome.

44. The method of claim 41, further comprising:
determining long-read nucleic acid fragment sequence data (second LRS data) from a second sample;
aligning records of the second LRS data to determine a second contig, wherein the second contig represents a chromosome or a plasmid of a microorganism in the second sample;
aligning the second contig with a second draft genome by correcting alignment errors of records of the first LRS data to construct a second consensus genome; and
comparing the second consensus genome with a database of known sequences of interest to detect sequences of interest in the second consensus genome.

45. The method of claim 44, further comprising comparing the sequences of interest detected in the first consensus genome with the sequences of interest detected in the second consensus genome to detect a horizontal transfer of any one of the sequences of interest between microorganisms in the first and the second sample.
